(19) United States
(12) Patent Application Publication
(10) Pub. No.: US 2015/0306035 A1
(43) Pub. Date: Oct. 29, 2015

(54) PHARMACEUTICAL COMPOSITION CONTAINING CONVENTIONAL LIPOSOMES AND PROLONGED-CIRCULATION LIPOSOMES FOR THE TREATMENT OF VISCERAL LEISHMANIASIS

(71) Applicants: UNIVERSIDADE FEDERAL DE MINAS GERAIS- UFMG, Belo Horizonte (BR); FUNDAÇAO DE AMPARO A PESQUISA DO ESTADO DE MINAS GERAIS- FAPEMIG, Belo Horizonte (BR)

(72) Inventors: Frederic JEAN GEORGES FREZARD, Belo Horizonte (BR); Erly GUILHERME AZEVEDO, Belo Horizonte (BR); Raul RIO BIBEIRO, Cruz das Almas (BR); Cynthia PERES DEMICHELI, Belo Horizonte (BR); Sydnei MAGNO DA SILVA, Belo Horizonte (BR); Simone APARECIDA REZENDE, Ouro Preto (BR)

(21) Appl. No.: 14/384,155

(22) PCT Filed: Mar. 11, 2013
(86) PCT No.: PCT/BR2013/000079

§ 371 Date: Dec. 24, 2014

(30) Foreign Application Priority Data
Mar. 9, 2012 (BR) BR1020120052652
Mar. 8, 2013 (BR) BR1020130056014

(51) Int. Cl. A61K 9/127; A61K 31/29

(52) U.S. Cl. CPC A61K 9/1271; A61K 31/29

(57) ABSTRACT

The present invention relates to a pharmaceutical composition for treating visceral leishmaniasis, characterized in that it comprises the association of conventional liposomes and prolonged-circulation liposomes with a leishmanicidal-drug delivery system. Said composition may be used in the preparation of a medicinal product for treating leishmaniasis and may be administered intramuscularly, subcutaneously, intraperitoneally or intravenously. The use of this system enables the drug efficiently to reach all sites infected by the parasite, the pegylated liposomes promoting more effective targeting of the leishmanicidal drugs on the bone marrow and spleen, whilst the conventional liposomes contribute to the targeting of the drug on the liver.

The present invention relates to a pharmaceutical composition for the treatment of visceral leishmaniasis, characterized by comprising the association of conventional liposomes and prolonged-circulation liposomes as a leishmanicidal-drug delivery system. This composition can be used in preparing a medicament for the treatment of leishmaniasis, and may be administered by intramuscular, subcutaneous, intraperitoneal or intravenous route. The use of this system enables the drug efficiently to reach all the sites infected by the parasite. The pegylated liposomes promote more effective targeting of leishmanicidal drugs to the bone marrow and the spleen, whereas conventional liposomes act on directing the drug to the liver.

Leishmaniasis are parasite diseases that affect about 12 million people all over the world, being caused by flagellate protozoans belonging to the order Kinetoplastida, family Tripanosomatidae and genus Leishmania (UNDP/World Bank/WHO Special Programme for Research & Training in Tropical Diseases (TDR). In Tropical Disease Research Programme Report, 13; progress 1995-96; World Health Organization: Geneva, 1997, chap. 8]. In Brazil, all recent data report the occurrence of about 30,000 new annual cases of the disease.

Leishmaniasis are transmitted to vertebrate hosts by the sting of an inset that regurgitates the parasite in the promastigote form. These parasites are phagocytized by macrophages, where they change into amastigotes. Amastigotes multiply freely in the acidic compartment of phagolisomes and escape from the defense systems of the host. Leishmania corresponds to a complex of various different species that cause various types of clinical manifestations, which include cutaneous, mucocutaneous and visceral forms. In Brazil, the species that causes visceral leishmaniasis is Leishmania chagasi. Dogs appear as the vertebrate host of the cutaneous and visceral forms and, particularly in the case of visceral leishmaniasis, they play an important role as reservoir and source of infection of the disease in an endemic area. Visceral leshmaniasis exhibits lethality rate of 100% in cases that are not treated clinically.

Starting in the 1940's, pentavalent antimony complexes (SB(V) began to be used in the therapy of Leishmaniasis [Marsden, P. D.; Rev. Soc. Bras. Med. Trop. 1985, 18,187; Berman, J. D.; Clin. Infect. Dis, 1997, 24, 684; Rath, S.; Trivelin, L. A.; Imbrunito, T. R.; Tomazela, D. M.; de Jesus, M. N.; Marzal, P. C.; Junior H. F. A.; Tempone, A. G.; Quim. Nova 2003, 26, 550]. The main antimonials in use at presente are Sb(V) complexes with N-methyl-glucamine (meglumine antimoniate) and with sodium gluconate (sodium stibogluconate). It was suggested that Sb(V) was a pro-drug, being reduced in the hos organism to Sb(III), which was said to be the active and toxic form. Although pentavalent antimonials continue to be the first-choice medicaments in the treatment of all the forms of leishmaniasis, the clinical use thereof has various limitations. These compounds should be administered by parenteral route (intravenous or intramuscular injection), daily, in a period of 20-40 days. In this context, the side effects are frequent. The appearance of cases of resistance to the treatment also represents a serious problem in the therapy of leishimaniases. Another difficulty encountered in controlling visceral Leishmaniasis is that so far there is no effective treatment for naturally infected dogs.

Among the new medicaments that might replace pentavalent antimonials are: (1) AmBisome®, a formulation of amphotericin B based on liposomes that was recently approved by the US Food and Drug Administration (FDA) for the treatment of visceral leishmaniasis [Meyerhoff A. 1999 U.S. Food and Drug Administration approval of AmBisome (liposomal amphotericin) for treatment of visceral leishmaniasis. Clin Infect Dis 28, 42-48]; (2) L'Impavido®, which is the hexadecilphosphocolin or mitephosine, initially developed under the name of Miltex® for the treatment of cancer and which exhibited high efficacy by oral route in the treatment of visceral leishmaniasis in clinical tests in India [Sundar S, Jha T K, Thakur C P, Engel J, Sendermann H, Fischer C, Junke K, Bryceson A, Berman J. 2002 Oral miltefosine for Indian Visceral leishmaniasis. N Engl J Med 347, 1739-1746]; and (3) a topical formulation of paromomycin (or aminosidin), which proved to be effective in the experimental treatment of cutaneous leishmaniasis [Gamier T, Croft S L. 2002 Topical treatment for cutaneous leishmaniasis. Curr Opin Investig Drugs 3, 538-544]. However, the prospecto f use of these medicaments is remote, due to the high oust of the AmBisome®, the side effects of Miltefosina and the low efficacy of the paromomycin formulation.

In view of these limitations, the World Health Organization recommends, also with incentives to other related entities like TDR (Special Programm for Research and Training in Tropical Diseses), the research on new medicaments [Remme, J. H. F.; Blas, E.; Chitsulo, L.; Desjeux, P. M. P.; Engers, H. D.; Kanyok, T. R.; Kayondo, J. F. K.; Kioy, D. W.; Kumaraswami, V.; Lasdins, J. K.; Nunn, P. P.; Oduola, A.; Ridley, R. G.; Toure, Y. T.; Zicker, F.; Morel, C. M.; Trends Parasitol. 2002, /8, 421; Ridley, R. G. Em Drug against Parasitic Diseases; Failamb, A. H.; Ridley, R. G.; Vial, H. J., eds.; UNDP/World Bank/WHO Special Programme for Research and Training in Tropical Diseases (TDR): Geneva, 2003, p. 13].

Essentially, three different strategies are being studied at present for improving the therapy of visceral leishmaniasis. One of them consists in combining different liehmanicidal agents with synergistic action. For instance, the association of gentamicin to paromomycin has increased the efficacy of paromomycin in topical application (WO9406439-A1). The combination of aminosidin with sodium stibogluconate, in turn, proved to be an effective measure in the treatment of the visceral form which does not respond to the conventional treatment. Similarly, the association of this antimonial with another drug in clinical evaluation, namely allopurinol, proved to be effective even in cases of resistance to antimonials [Martinez, S.; Gonzalez, M.; Vernaza, M. E.; C/in Infect Dis 1997, 24, 165]. Immunochemotherapy, which associates immunomodulating substances to pentavalent antimonials, proved to be a way to reduce the applied dose of antimonial, keeping the efficacy of the treatment [Murray, H. W.; Berman, J. D.; Wright, S. D.; J Infect Dis 1988, 157, 973; Machado-Pinto, J.; Pinto, J.; da Costa, C. A.; Genaro, O.; Marques, M. J.; Modabber, F.; Mayrink, W.; Int. J. Dermatol. 2002, 41, 73].

A second strategy involves the planning/synthesis of new active substances or of known drugs, with chemical modifications. However, the long time and high cost of the development of a new drug limit considerably the success of this strategy.

The third strategy involves the reversible strategy of drugs already in use to a carrying system, with a view to lead the drug to a target cell in a better manner and prevent the undesirable sites where the drug exerts toxicity. This strategy, known also as “rejuvenation of drugs”, provides a gain in time in the phase of developing the product, since it uses a drug that is already characterized from the pharmacological point of view. Among the medicament carrying systems available at present, liposomes occupy an outstanding position for the therapy of leishamaniasis forms.

The use of liposomes as drug carriers has been a tendency in the pharmaceutical industry and has been opening prospects for the development of new leishmanicidal medicaments. These spherical vesicles, constituted by one or more concentric bi-layers of lipids, can store, in their internal aqueous compartment, water-soluble active principles, or have lipophilic or amphiphilic active principles incorporated in their membranes, Thus, the medicament is released slowly, which prevents the rapid elimination thereof by the organism. The result is an increase of the bioavailability of the medicament, with potentiation of the action and reduction of toxicity.

Conventional liposomes were widely studied for carrying leishmanicidal drugs, aiming at the treatment of visceral leishmaniasis [Frézard, F., Demicheli, F. 2010. New delivery strategies for the old pentavalent antimonial drugs. Expert Opin. Drug Deliv. 7(12), 1343-58]. Conventional liposomes are typically formed from a phospholiopide such as phosphatidylcolin, or from a non-ionic surfactant. The may also include, in its composition, cholesterol, a phospholipide with a negative charge, as for example, phosphatidylglycerol, phosphatidic acid, dicetylphosphate and/or a phospholipide with a positive charge, such as sterarylamine. Since they are rapidly cleared from the blood circulation by the macrophages of the mononuclear phagocytic system, mainly the liver, the spleen and the bone marrow, the conventional liposomes carry the drug to the sites of infection of visceral leishmaniasis, which makes available a larger amount of the drug to interact with the parasite.

In this context, an amphotericin B formulation was developed in conventional liposomes (AmBisome®) [WO9640060-a1), which was used successfully in the treatment of patients who did not respond to antimonial drugs, and the same thing happened in the treatment of patients with the PKDL form, without report of side effects. The efficacy of 100% on immunocompetent patients earned it the approval by the US Food and Drug Administration (FDA) with the first presentation based on liposomes to be recognized for the treatment of Calazar (visceral leishmaniasis) [Meyerhoff A. 1999 U.S. Food and Drug Administration approval of AmBisome (liposomal amphotericin) for treatment of visceral leishmaniasis. Clin Infect Dis 28, 42-48].

In the case of the antimonial compounds, encapsulated forms in conventional liposomes were also developed [U.S. Pat. No. 4,186,183A; EP72234A; W09604890-A1; U.S. Pat. No. 4,594,241]. In an experimental model of visceral leishmaniasis, these preparations proved to be at least 200 times as effective as the non-encapsulated antimonial in eliminating the parasite in the liver [New, R. R.; Chance, M. L.; Thomas, S. C.; Peters, W.; Nature 1978, 25 272(5648), 55; Alving, C. R.; Steck, E. A.; Chapman, W. L.; Waits, V. B.; Hendricks, L. D.; Swartz, G. M.; Hanson W. L.; Proc. Natl. Acad. Sci. U.S.A. 1978, 75, 2959; Black, C. D. V.; Watson, G. J.; Ward, R. J.; Trans. Roy. Soc. Trop. Med. Hyg. 1977, 71, 550]. Studies on biodistribution/pharmacokinetics of antimony on mice and dogs showed that the conventional liposomal form promotes much higher and prolonged levels of antimony and in the liver and in the spleen of the animals, as compared with the free form [Collins, M., Carter, K. C., Baillie, A. J., O'Grady, J.; J. Drug Targeting 1993, 1, 133; Frezard e Demicheli. 2010 New delivery strategies for the old pentavalent antimonial drugs. Expert Opin Drug Deliv 7, 1343-58]. However, the low stability of these first formulations explains, at least in part, that none of them came to be marketed.

More recently, efforts were made with a view to obtain novel formulations of the meglumine antimoniate in conventional liposomes with more favorable technological characteristics. Thus, a novel preparation process was developed which consist in re-hydrogenating lyophilized empty liposomes formed by phosphatidilcoline, cholesterol and dicetylphosphate with an antimonial solution [Frézard F, Michalick M S M, Soares C F, Dermicheli C. 2000. Novel methods for the encapsulation of meglumine antimoniate in liposomes. Braz J. Med Biol Res 33, 841-6]. A significant technological advantage of the method of preparing this formulation, as compared to convention methods, is that the resulting formulation may be stored in the form of lyophilized empty liposomes, and the re-hydration is carried out shortly before administration, which facilitates the preservation and transport thereof.

A single endovenous injection of the liposomal formulation into healthful dogs resulted in high levels of antimony in the liver and in the spleen, corresponding to about 40% of the injected dose, for a period of 4 days [Schettini D A, Costa Val A P, Souza L F, Demicheli C, Rocha O G F, Melo M N, Michalick M S M, Frezard F. 2003. Distribution of liposome-encapsulated antimony in dogs. Braz J Med Biol Res 36, 269-72]. On the other hand, the levels of antimony in the bone marrow were much lower, about 10 times as low. Besides, after treatment of dogs naturally infected with multiple doses of this formulation, a significant reduction in the number of parasites in the bone marrow was observed, but there was no complete elimination of the parasites in that tissue [Schettini D A, Costa Val A P, Souza L F, Demicheli C, Rocha O G F, Melo M N, Michalick M S M, Frezard F. 2005. Pharmacokinetic and parasitological evaluation of the bone marrow of dogs with visceral 30 leishmaniasis submitted to multiple dose treatment with liposome-encapsulated meglumine antimoniate. Braz J Med Biol Res 38, 1879-83]. These data suggested that the increase in the concentration of antimony in the bone marrow is probably critical to obtain the cure with this formulation. On the basis of the prior art [Carter, K. C.; Dolan, T. F.; Alexander, J.; Baillie, A. J.; McColgan, C.; J. Pharm. Pharmacol, 1989, 41, 87], one raised the hypothesis that the large size of these conventional liposomes (average hydrodynamic diameter larger than 1000 nm) might be responsible for the low routing of the vesicles to the bone marrow.

Thus, in a subsequent study, the process of preparing the above-mentioned liposomes was modified by introducing a cryoprotector sugar, so as to obtain liposomes of reduced size [Frezard F, 10 Demicheli C, Schettini D A, Ribeiro R R, Melo M N, Michalick M S M. A process for the preparation of pharmaceutical formulations from meglumine antimoniate in liposomes and use of the pharmaceutical formulations on animals attacked by visceral leishmaniasis. Patent application filing at the INPI PI0405489-0, Nov. 11, 2004]. The use of sucrose in the sugar/lipid ratio 3:1 (p/p), enabled the average diameter of the vesicles to be reduced from 1200 nm to 400 nm. The two formulations of different sizes were administered to naturally infected dogs in the same doses of lipid and of antimony, and were compared by capability of routing the antimony to the bone marrow. The “medium” liposomes (average diameter of 400 nm) promoted, in this tissue, level of antimony 3 to 4 times as high as those achieved with the “big” liposomes (average diameter of 1200 nm) [Schettini D A, Ribeiro R R, Demicheli C, Rocha O G F, Melo M N, Michalick M S M, Frezard F. 2006. Improved targeting of antimony to the bone marrow of dogs using liposomes of reduced size. Int J Pharm 315, 140-7]. This result is of great interest, since it suggests higher efficacy of the reduced-size liposome formulation for eliminating the parasite in the bone marrow of the animals.

In more recent studies [Ribeiro R R, Moura P, Pimentel V M, Sampalo W M, Silva S M, Schettini D A, Alves C F, Melo F A, Demicheli C, Tafuri W L, Melo 30 M N, Frezard F, Michalick M S M. 2008 Reduced tissue parasitic load and infectivity to sand flies in dogs naturally infected by Leishmania (Leishmania) chagasi following treatment with a liposome formulation of meglumine antimoniate. Antimicrob Agents Chemother 52, 2564-72], one evaluated the therapeutic efficacy and the toxicity of formulation of the meglumine antimoniate in liposomes of medium size on dogs naturally infected by Leishmania chagasi. Groups of 12 animals received, by endovenous route, 4 doses (with intervals of 4 days), either of the meglumine antimoniate encapsulated in liposomes (GI, 6.5 mg Sb/kg/dose), of empty liposomes (GII) or of saline solution (GIII). Parameters marking the renal, hepatic and hematopoietic functions, when evaluated before and right after the treatment, did not exhibit significant alterations, indicating absence of toxicity of the formulation. The parasite load in the bone marrow of the GI showed a significant reduction 4 days after the treatment, as compared to that of the control groups. Immunocytochemical evaluations of the parasite load in the skin, in the lymphnodes, in the liver, in the spleen and in the bone marrow, 5 months after treatment, showed a significant reduction of the lymphnodes, in the liver and in the spleen of the animals of the GI, as compared with the animals of the GII and GIII. When flebotomides Lutzomya (Lutzomyia) longipalpis were fed to the animals in the different groups 5 months after treatment, a significantly lower infection rate was found in GI, as compared with groups GII and GIII. It should be pointed out that this high therapeutic efficacy was achieved by using a cumulated dose of antimony 25 times as low as that used in the conventional treatment.

However when cultures of bone marrow of the animals of GI were carried out 5 months after treatment, the presence of the parasite was evidenced in all the animals.

It should be pointed out that the treatment with AmBisome was not capable of curing dogs naturally infected with Leishmania infantum either [Oliva G, Gradoni L, Ciaramella P, et al. Activity of liposomal amphotericin B (AmBisome) in dogs naturally infected with Leishmania infantum. J Antimicrob Chemother 1995; 36:1013-19].

Therefore, the prior art shows that the technologies based on conventional liposomes, available at present for providing the routing of the drug to the infection sites of affected animals, mainly dogs with visceral leishmaniasis, do not enable one to achieve sufficient levels of the drug to eliminate the parasite at determined infection sites. More specifically, the bone marrow seems to be a critical tissue, in which it is necessary to increase the concentration of the drug.

In the Nineties, different researchers simultaneously reported that prolonged-circulation liposomes can be obtained by incorporating into the membrane of the vesicles a lipid with polar head constituted of ethylene glycol polymer (PEG). In this case, this new class of liposomes was called pegylated, furtive or sterically-stabilized liposomes [Klibanov e Col. 1990. FEBS Letter 268:235-237; Allen e Col. 1991. Biochim Biophys Acta 1066, 29-36; Woodle M C, Lasic D D. 1992 Sterically stabilized liposomes. Biochim Biophys Acta 1113, 171-99]. A non-limiting example of these lipids is di-stearoylphosphatidylethanolamine-PEG (2000) (DSPE-PEG200), incorporated typically in a proportion of 5 to 10 mole % of the total lipids.

This chemical modification of the vesicle surface has the following impacts on the pharmacokinetics thereof [Frezard F, Schettini D A, Rocha O G F, Demicheli C. 2005 Liposomes: physicochemical and pharmacological properties, applications in antimony-based chemotherapy. Quim Nova 28, 511-20 518]: reduction of the level of opsonization of the liposomes; reduction of the velocity of capturing by the macrophages of the organs of the phagocytic mononuclear system (liver, spleen, bone marrow); prolonged circulation in the blood plasma; preferred accumulation at inflammation/infection sites due to the fact that these sites exhibit increased vascular permeability. This property is being exploited in the development of radiomarked liposomes for diagnose of inflammation/infection sites [Boerman e Col. 1997. Optimization of technetium-99m-labeled PEG liposomes to image focal infection: effects of particle size and circulation time. J Nucl Med 38, 489-493].

Studies in this area have established that the plasmatic half-life of liposomes containing PEG lipid depends on various factors. The ideal characteristics of the pegylated liposomes are: i) an average diameter of the vesicles ranging from 150 to 200 nm; ii) the use of a PEG having molecular weight of about 2000 Da; iii) incorporation of the PEG-lipid in the relation of 5 mole % with respect to the other lipids [Woodle M C, Lasic D D. 1992 Sterically stabilized liposomes. Biochim Biophys Acta 1113, 171-99].

Other chemical entities that may replace PEG and impart prolonged-circulation properties to the liposomes are polyvinylpyrrolidine, polyethyloxazolin, polyethyl-oxazolin, polyhydroxypropyl-metacrylamide, polylactic acid, polyglycolic acid and derivatized celluloses, such as hydroxymethylcellulose or hydroxyethylcellulose [Patente U.S. Pat. No. 7,666,674]. The use of lipids having partly fluorinated or perfluoroalkylated hydrophobic chains is another strategy that enables one to prolong the plasmatic half-life of the liposomes [Santaella C, Frezard F, Vierling P, Riess J G. 1993 Extended in vivo blood circulation time of fluorinated liposomes. FEBS Lett. 336:481-4].

Considering that the liver, the spleen and the bone marrow are the main infection sites in visceral leishmaniasis and that these tissues play a fundamental role in removing the liposomes from the blood circulation, the plasmatic half-life and the distribution of the pegylated liposomes after administration to affected animals cannot be predicted a priori, since the pathologic state may affect the liposome opsonization process and the recognition thereof by the tissue macrophages. In this regard, the efficacy of the camptotecin encapsulated in pegylated liposomes, as compared to its free form, exhibited a significant improvement with regard to the parasite load in the liver, in murine model of visceral leishmaniasis [Proulx, M.-E., Desormeaux, A., Marquis, J.-F., Olivier, M., Bergeron M. G. 2001 Treatment of Visceral Leishmaniasis with Sterically Stabilized Liposomes 25 Containing Camptothecin. Antimicrob Agents Chemother 45, 2623-7], suggesting that the pegylated liposomes do not accumulate in the liver and are little promising alone, for treating visceral leishmaniasis.

The present invention relates to a pharmaceutical composition for the treatment of visceral leishmaniasis, characterized by comprising the association of conventional liposomes and prolonged-circulation liposomes as a leishmanicidal-drug delivery system. This composition can be used in preparing a medicament for the treatment of the forms of leishmaniasis, and may be administered by intramuscular, subcutaneous, intraperitoneal or intravenous route. The use of this system enables the pegylated liposomes to promove the more effective routing of leishmanicidal drugs to the bone marrow and the spleen, whereas the conventional liposomes act in routing the drug to the liver.

According to the present invention, after administration to dogs naturally affected by visceral leishmaniasis, pegylated liposomes containing encapsulated meglumine antimoniate promote a longer half-life ion the blood than the medium-size (medium size of 410 nm) and reduced-size (medium size of 175 nm) (Table 2) conventional liposomes, which establishes the property of prolonged circulation of pegylated liposomes in infected dogs and the potential thereof to distribute the drug to tissues other than the liver, After evaluation of the distribution of antimony in the liver, in the spleen and in the bone marrow 24 hours after endovenous administration of each formulation, the pegylated liposomes promoted a more effective routing to the bone marrow, whereas conventional liposomes of medium size (medium size of 410 nm) resulted in higher levels of antimony in the liver of the animals (Table 3).

The set of this results validated in naturally infected dogs the model in which the pegylated liposomes enable one to achieve a higher concentration of antimony at infection sites other than the liver and that the conventional liposomes of medium size enable one to achieve a higher concentration of antimony in the liver.

According to the present invention, the composition characterized by the mixture of the two types of liposomes, that is, conventional liposomes and pegylated liposomes encapsulating meglumine antimoniate was more effective in eliminating parasites in the spleen of mice experimentally infected with Leishmania infantum chagasi, as compared with the compositions of each type of liposomes administered separately (Table 4 and FIG. 2). On the other hand, all the compositions exhibited the same efficacy in reducing the parasite load in the liver. Therefore, these results prove, for the first time, the superiority of the mixture of the two types of liposomes as compared with each type of liposomes, in reducing the parasite load in an infection site other than the liver.

Besides, the present study reports, for the first time, the efficacy of pegylated liposomes and of the mixture thereof with conventional liposomes in reducing the parasite load in the bone marrow of mice (FIG. 3). These results differ from those achieved in the literature with liposomes and niosomes containing antimonial, which did not prove to be capable of reducing the parasite load in this tissue (Carter K C, Baillie A J, Alexander J, Dolan T F. 1988 The therapeutic effect of sodium stibogluconate in BALB/c mice infected with Leishmania donovani is organ-dependent. J Pharm Pharmacol 40, 370373).

The proposed innovation differs from the existing technologies, which consist of conventional liposomes [U.S. Pat. No. 4,186,183A; EP72234A; W09604890-A1; U.S. Pat. No. 4,594,241 BR0405489] or of prolonged circulation liposomes [Proulx e Col. 2001 Antimicrob. Agents Chemother 45, 2623-7]. The new technology is more effective than those based on conventional liposomes, since it increases the distribution of the drug to other infection sites besides the liver. It is also more effective than those based in prolonged circulation liposomes, since it enables one to achieve a high concentration and action of the drug in the liver.

The proposed technology may be applied in the treatment of canine and human visceral leishmaniasis, resulting in a more effective and safe treatment. A greater adhesion of the patients to this treatment is also expected as a function of the lower incidence of side effects (because of the smaller total dose of drug administered), of the smaller number of doses and of the longer interval between the doses. It also opens new prospects for the therapeutics of visceral leishmaniasis, with the possibility of achieving parasitologic healing on naturally infected dogs.

### BRIEF DESCRIPTION OF THE FIGURES

FIG. 1: Parasite load in the liver of BALB/c mice infected with the Leishmania (Leishmania) infantum 043 strain, determined by the limiting dilution technique 8 seeks after infection of the animals and 2 weeks after treatment with the different formulations in single dose by endovenous route (n=8). *p<0.05 (Kruskal-Wallis test) for comparison between the groups that received liposomes containing encapsulated meglumine antimoniate (MA/AM) and the control group (saline and empty liposomes) (n=8).

FIG. 2: Parasite load in the spleen of BALB/c mice infected with the Leishmania (Leishmania) infantum 043 strain, determined by the limiting dilution technique 8 weeks after infection of the animals and 3 weeks after treatment with the different formulations in single dose by endovenous route (n=8). * p<0.05 (Kruskal-Wallis tet) for comparison between the groups that received encapsulated AM/MA the control groupo (saline). # p<0.05 (Kruskal-Wallis test) for comparison with the group MIX 200+4.7 with groups Lconv 200 and Lpeg 4.7 ( )n=8).

FIG. 3: Parasite load in the bone marrow of BALB/c mice infected with the Leishmania (leishmania) infantum 043 strain, determined by the limiting dilution technique 8 weeks after infection of the animals and 2 weeks after treatment with the different formulations in single dose by endovenous route (n=8). * p<0.05 (Kruskal-Wallis test) for comparison with groups Lpeg 4.7 and MIX 200+4.7 and the control groups (saline and empty liposomes). # p<0.05 (Kruskal-Wallis test) for comparison with group MIX 200+4.7 WITH GROUP Lconv 200 (n=8).

### DETAILED DESCRIPTION OF THE TECHNOLOGY

The present invention relates to a drug composition for the treatment of visceral leishmaniasis, characterized by comprising the association of conventional liposomes and prolonged-circulation liposomes with a leishmanicidal-drug delivery system. This composition can be used in preparing a medicament for the treatment of the leishmaniasis forms, and may be administered by intramuscular, subcutaneous, intraperitoneal or intravenous route. In this composition, the conventional liposomes may comprise natural or synthetic phospholipids and/or surfactants and/or cholesterol and have an average hydrodynamic diameter ranging from 20 to 1000 nm.

In this composition, the prolonged-circulation liposomes can be obtained by including in the lipid composition liposomes of one lipid or surfactant that results in the increase of the time of circulation of the liposomes in the blood stream. A non-limiting example is given by lipids or surfactants with polar head constituted by ethyleneglycol polymer (PEG), preferably di-stearoylphosphatidylethanolamine-PEG (5000).

Leishmaniacidal drug (s) may be selected from the group consisting of drugs in clinical use at present, including antimony complexes, amphotericin B, pentamidine, miltefosine, alopurinol and paramomycin, but also from the group of phamaceuticals with leishmanicidal activity, established experimentally, including sitamaquine, iminoquimod, fluconazole, cetoconazol, itraconazol, posaconazol, tucaresol, azitromicin, buparvaquona, tamoxifen, terbinafin, furazolidone, fluoroquinolone, domperidone, interleukin 12, interleukin 10, lipid A, derivatives of alkyl-lisophospholipid and of alkyl-phospholipide, azasterols, lichocalcone A, maesabadid Ill, trichothecenes, n-acetyl-cistein, 3-substituted quinolin [Monzote L. 2009 The Open Antimicrobial Agents Journal 1, 9-19].

The invention described herein can be better understood by means of the following non-limiting examples.

### Example 1

### Preparation and Characterization of Suspensions of Conventional Liposomes and Pegylated Liposomes Containing the Encapsulated Meglumine Antimoniate

A lipid film was formed from the di-stearoylphosphatidylcholine (DSPC), cholesterol (COL) and dicetylphosphate (DCP) at the mole ratio 5:4:1 (conventional liposomes) or from DSPC/COL/DCP/di-stearoylphosphatidylethanolamine-PEG(2000) at the mole ratio 4.53:1:4:0.47 (pegylated liposomes). The film was hydrated with distilled water at a temperature of 60° C. and subjected to mechanical stirring, which led to the obtainment of multilamelar-type liposomes.

Then, the resulting multilamelar-lipossome suspension was subjected to ultrasonication (Probe-type Ultrasonic Processor, 500 Watts) at a temperature of 60° C., so as to obtain small uni- and oligo-lamelar liposomes. The resulting suspension was then filtered by using sterile filters having pores of 0.2 μm in diameter. To this suspension an aqueous solution of cryoprotective sugar was added at sucrose/lipid ratio of 1:1 (m/m) (pegylated liposomes and calibrated conventional liposomes) or 3:1 (m/m) (non-calibrated conventional liposomes). Right afterward, this mixture was frozen by immersion into liquid nitrogen and subjected to lyophilization (freeze-dryer, Labconco). At this stage, the lyophilized formulation may be stored at −20° C. for at least 6 months without impairing the final characteristics of size and encapsulation rate of the formulation.

One day prior to the administration, the lyophilizate was then hydrated with an aqueous solution of the meglumine antimoniate at the concentration of antimony of 80 g/L and using a mass ratio Sb/lipid of about 0.6. In this phase of hydration, the suspension was incubated for 30 min at 60° and subjected to mechanical stirring (use of a vortex) in the time 0 and every 10 min.

In the case of pegylated liposomes and of calibrated conventional liposomes, the liposome suspension was subjected to the extrusion process by repeated filtrations at 65° C. through polycarbonate membranes with pores having a diameter of 200 nm (Extruder, Lipex biomembrane, Canada). After this step, the liposome suspension was diluted with the sterile saline solution (NaCl 0,9% (m/v)) and subjected to centrifugation at 20,000×g for 40 min at 4 C. The supernatant of the centrifugation was eliminated and the liposome precipitate was washed twice in a sterile saline solution (re-suspension in a sterile saline solution and centrifugation at 20,000×g for 40 min at 4° C.). The liposome precipitate was finally re-suspended in the desired volume of sterile saline solution, typically at the concentration of 10 g/L antimony.

For determination of the encapsulated antimony rate, the sample was subjected to digestion with nitric acid and the antimony was dosed by graphite furnace atomic absorption spectrometry (ETASS).

The liposome size was determined by photon correlation spectrometry by using a particle size analyzer (Malvern Instrument, UK).

The results achieved, with antimony encapsulation rates and the average diameters of liposomes, are shown in Table 1 below.

TABLE 1
Characteristics of conventional liposomes and pergylated
liposomes with respect to the meglumine antimoniate
encapsulation and to the vesicle size

| | | Average | |
|---|---|---|---|
| | Encapsulation | diameter of the | Polydispersion |
| Liposome | rate (%) | vesicles (nm) | rate |
| Conventional | 40 | 410 | 0.3 |
| liposomes (non- | | | |
| calibrated) | | | |
| Conventional | 34 | 175 | 0.07 |
| liposomes | | | |
| (calibrated | | | |
| Pegylated | 16 | 154 | 0.09 |
| liposomes | | | |

Therefore, the present example shows simple processes for obtaining suspensions of conventional liposomes and of pegylated liposomes containing encapsulated meglumine antimoniate.

### Example 2

### Plasmatic Half-Life and Concentrations of Antimony in the Liver, in the Spleen and in the Bone Marrow of Dogs with Visceral Leishmaniasis after Endovenous Administration of the Meglumine-Antimoniate Formulations in Conventional and Pegylated Lipossoms

With a view to determine and compare the plasmatic half-life of conventional liposomes and of prolonged-circulation liposomes, as well as their capability of routing an encapsulated drug to the spleen, liver and bone marrow of animals with visceral leishmaniasis, the suspensions of liposomes containing encapsulated meglumine antimoniate were prepared as described in Example 1 and administered in single dose by endovenous route to dogs naturally infected with L. chagasi.

Three groups of dogs of indefinite race, weight 5 to 15 kg, with proven infection by L. chagasi, were used in this experiment. The first group (3 animals) received by endovenous rout the formulation of non-calibrated conventional liposomes at the dose of 4.2 mg of Sb/kg of body weight. The second group (5 animals) received by endovenous the formulation of calibrated conventional liposomes at the dose of 6.5 of Sb per kg of body weight. The third group (4 animals) received by endovenous route the formulation of pegylated liposomes at the dose of 3.7 mg of Sb per kg of body weight.

In the period of 24 hours after administration, blood samples were taken at different intervals of time for antimony dosage by graphite furnace atomic absorption spectrometry (GFAAS). In the period of 24 hours the dogs were sacrificed and samples of the liver, spleen and bone marrow were removed. The liver and spleen were weighed and homogenized. The tissue samples were then subjected to digestion with nitric acid and taken for later determination of antimony by atomic absorption spectrometry in a graphite furnace. The results of half-life of antimony in the blood are shown in Table 2. The results of percentages of antimony dose recovered in the organs are shown in Table 3.

TABLE 2
Half-life of antimony in the blood of dogs affected by visceral
leishmaniasis, after endovenous administration of the
formulations of conventional and pegylated liposomes
Half-life of antimonyin the blood of doss with LV

| After administration of | | After administration of pegylated |
|---|---|---|
| conventional liposomes | | lipososomes |
| Non- | Calibrated (175 nm) | 224 min |
| calibrated | | |
| (410 nm) | | |
| 27 min | 128 min | |

TABLE 3
Percentage of the administered dose of antimony found
in the liver, in the spleen and in the bone marrow of
dogs affected by visceral leishmaniasis, 24 hours after
endovenous administration of the formulations of
conventional and pegylated liposomes (data shown as
average ± DP).

Percentage of antimony (%)

| | |
|---|---|
| Conventional liposomes | Pegylated |

| Tissue | Non-calibrated | Calibrated | liposomes |
|---|---|---|---|
| Liver | 50.0 ± 8.7 | 7.7 ± 2.9 | 9.2 ± 1.2 |
| Spleen | 6.1 ± 0.8 | 3.3 ± 1.7 | 6.7 ± 6.0 |
| Bone | 1.4 ± 0.8 | 06. ± 0.4 | 2.0 ± 0.4 |
| marrow | | | |

These data show that conventional liposomes exhibit a short half-life, as compared with pegylated liposomes (Table 2). Therefore, the results achieved confirm that properties of prolonged circulation of pegylated liposomes in naturally infected dogs, as compared with conventional liposomes. Besides, conventional liposomes of medium size promote a greater routing of the antimony to the liver of the animals, whereas pegylated liposomes exhibit a greater routing to the bone marrow while keeping a high routing to the spleen (Table 3).

The set of these results validates, in naturally infected dogs, the model that pegylated liposomes enable one to achieve a higher concentration of antimony ion other infection sites than the liver, due to their prolonged-circulation characteristic, and that conventional liposomes of medium size enable one to achieve a much higher concentration of antimony in the liver.

The data obtained suggest that the mixture of conventional liposomes with pegylated liposomes would enable one to enhance the routing of antimony to the bone marrow and to guarantee the routing of the antimony to the liver and spleen of animals affected by visceral leishmaniasis. Therefore, by using this combination, one expects a more effective combat of visceral leishmaniasis.

It should be further pointed out that the endovenous injection of these pharmaceutical formulations, even with the high dose of antimony administered, was well tolerated in the animals.

### Example 3

### Antileishmania Activity of Formulations of Conventional liposomes and Pegylated Liposomes Containing Meglumine-Antimoniate and of the Association of the Two Types of Liposomes in a Murine Model of Visceral Leishmaniasis

BALB/c female mice were infected with L. ionfantum chagasi (M2682—MHM/BR/74/PP75 strain). 14 days after inoculation of the parasites through the caudal vein, the animals received different compositions of conventional liposomes and pegylated liposomes and mixtures thereof in single dose of 10 mg Sb/kg by endovenous route. The mixture consisted of conventional liposomes at the dose of 5 mg Sb/kg and pegylated liposomes at the dose of 5 mg Sb/kg. 14 days after treatment, the animals were killed and the liver and the spleen were taken for evaluation of parasite load by the limiting dilution technique.

Table 4 shows the proportions of negativated animals (without parasite detected) in the liver and in the spleen.

TABLE 4
Antileishmania activity of conventional liposomes and
of pegylated liposomes containing meglumine antimoniate
in BALB/c mice infected with Leishmania infantum chagasi.
The data shown are the proportions of negativated animals
(without parasite detected) in the liver and in the spleen
after treatment by endovenous route with single dose of
10 mg Sb/kg (n = 8 per group).

| | Proportion of | |
|---|---|---|
| | negativated animals | |
| Formulation medium diameter | Liver | Spleen |
| Non-calibrated conventional liposomes | 87.5%* | 0% |
| (410 nm) | | |
| Calibrated conventional liposomes (175 nm) | 100* | 12.5% |
| Pegylated liposomes (154 nm) | 87.5%* | 0* |
| Mixture of calibrated conventional | | |
| liposomes ( )5 mg Sb/kg) + pegylated | | |
| lisosomes (5 mg Sb/kg) | | |
| Empty non-calibrated conventional | 0% | 0% |
| liposomes | | |
| Empty calibrated conventional | 0% | 0% |
| liposomes | | |
| Saline | 0% | 0% |

*Significant difference with respect to the groups treated with formulations without antimony;
** - Significant difference with respect to the other groups (P < 005, exact Fisher test).

The results of Table 4 show that all the formulations exhibited equivalent efficacy in reducing the parasite load in the liver of the mice with respect to the control groups (treated with empty or saline liposomes). However, in the spleen only the formulation associating conventional liposomes and pegylated liposomes proved to be capable of reducing the parasite load significantly.

This data demonstrates clearly and in a surprising manner the synergism of the two types of liposomes (conventional and pegylated) for reducing the parasite load in the spleen of animals.

Therefore, these results prove, for the first, time, the superiority of the mixture of the two types of liposomes as compared with each type of liposome in isolation, in reducing the parasite load in an infection site other than the liver.

### Example 4

### Efficacy of the Formulations of Pegylated Liposomes and Conventional Lipossomes and of the Mixture Thereof in Reducing the Parasite Load in the Liver, in the Spleen and in the Bone Marrow, in a Murine Model of Visceral Leishmaniasis

The Leishmania (leishmania) infantum C43 strain, obtained from an isolate of symptomatic dog and characterized by RFLP, was used for its high capability of infecting the bone marrow of mice.

BALB/C mice (8 animals per group) were inoculated into the caudal vein with 1×10⁷ promastigote forms of Leishmania infantum chagasi. After six months, the animals received one of the following formulations in single dose by endovenous route;
1) Formulation of meglumine antimoniate (MA) in pegylated liposomes of 200 nm (Lpeg 4.7, at the dose of 10 mg Sb/kg);
2) Formulation of MA in conventional liposomes of 200 nm (Lconv 200, at the dose of 10 mg Sb/kg);
3) Formulation of MA constituted by the mixture of Lpeg 4.7 (5 mg Sb/kg) with Lconv 200 (5 mg Sb/kg) (MIX 200+4.7);
4) Formulation of empty conventional liposomes Lconv 200;
5) Formulation of empty pegylated liposomes Lpeg 4.7;
256) Phosphate buffer 10 mM containing NaCl 0.15 pH 7.4 (PBS).
   The MA formulations in liposomes were prepared and characterized as described in Example 1. The Sb and lipid concentrations were adjusted to 3.77 g/L and 49.8 g/L, respectively, with addition of PBS or of a suspension of empty liposomes of the same lipid composition and identical size. Table 5 shows the characteristics of size and rate of encapsulation of the formulations administered, as well as the adjustments made.

For the preparation of the liposome mixture, equal volumes of the formulations Lconv 200 and Lpeg 4.7 were mixed at the moment immediately preceding the administration of the formulation to the animals.

TABLE 5
Rate of antimony encapsulation and volume adjustments
made for the MA/AM formulations in liposomes for
efficacy experiment with the C43 strain

| | | Concentra- | Volume | Vol. | | |
|---|---|---|---|---|---|---|
| | Encap- | tion of Sb | (μL) | (μL) | Vol. of | Total |
| Formula- | sulation | in the lipos | Lipos. | empty | PBS | volume |
| tion | rate (%) | (mg/mL) | With Sb | lipos. | (μl) | (μL) |
| Lpeg 4.7 | 11 | 4.40 | 60.0 | — | 10 | 70 |
| Lconv 200 | 29 | 11.72 | 22.5 | 37.5 | 10 | 70 |

Two weeks after treatment (8 weeks after inoculation of the parasites), the mice were killed by cervical displacement, and the liver, spleen and bone marrow were collected for determination of the parasite load through the limiting dilution technique.

Table 6 shows the characteristics of liposome formulations administered in single dose to BALB/c mice infected with the L infantum chagasi 043 strain. All the formulations exhibited populations with monodispersed vesicles (polydispersion rate <0.3) with an average hydrodynamic diameter close to 200 nm.

TABLE 6
Characteristics of size and doses of Sb and of lipid
administered of different formulations of meglumine
antimoniate in liposomes.

| | | | Dose of Sb | Dose of lipid |
|---|---|---|---|---|
| | Diameter | Polydispersion | administered | administered |
| Formulation | (nm) | rate | (mg Sb/Kg) | (mg lipid/Kg) |
| Lconv 200 | 193 | 0.066 | 10 | 132 |
| Lpeg 4.7 | 208 | 0.084 | 10 | 132 |
| MIX 200 + | 205 | 0.090 | 10 | 132 |
| 4.7 | | | | |
| Empty | 205 | 0.035 | | 132 |
| Lconv | | | | |
| Empty Lpeg | 180 | 0.177 | | 132 |

The determination of the parasite load in the liver, spleen and bone marrow of the mice was made 8 weeks after inoculation of the parasites and 2 week after administration of the treatment and, in order to coincide with the peak of the parasite load in the bone marrow.

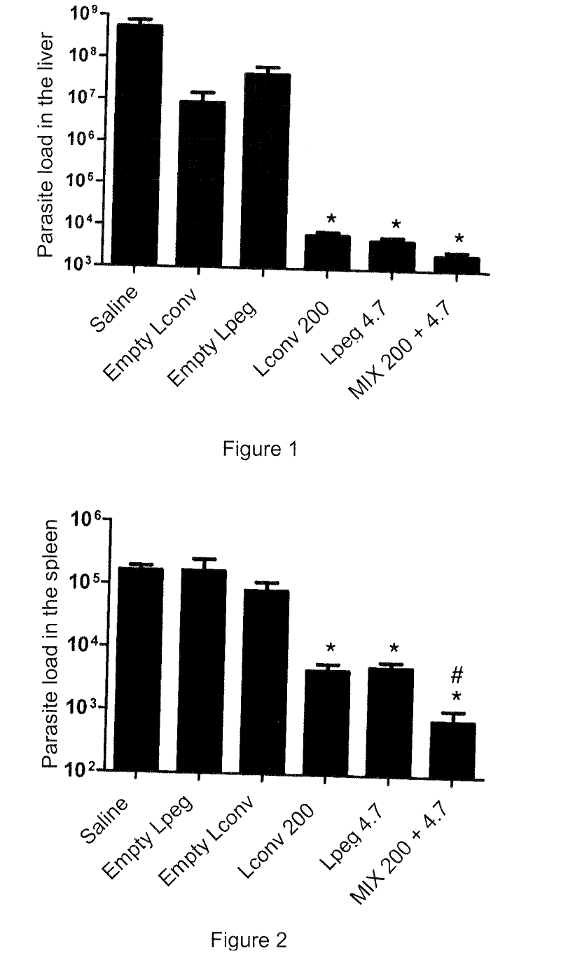

FIGS. 1, 2, and 3 show the parasite loads ion the liver, in the spleen and in the bone marrow, respectively.

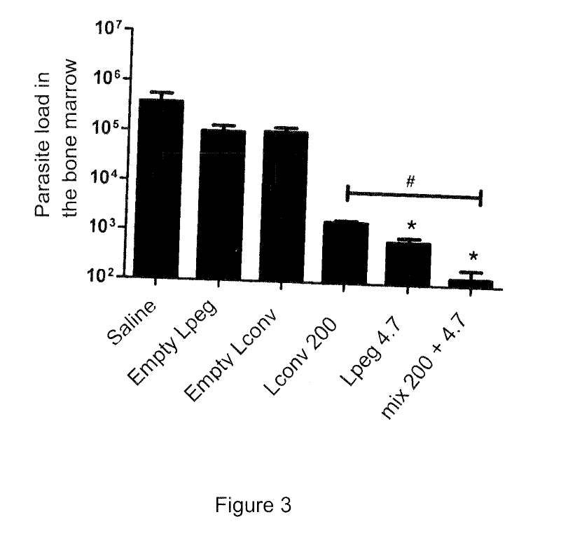

The results show that the MA formulation in pegylated liposomes and in the mixture of pegylated and conventional (MIX 200+4.7) promoted significant reductions of the parasite load in all the organs evaluated, as compared with the control treatment (saline or empty liposomes) (FIGS. 1, 2 and 3). On the other hand, the MA formulation ion conventional liposomes promoted a significant reduction of the parasite load in the liver (FIG. 1) and in the spleen (FIG. 2) of the animals, but not in the bone marrow (FIG. 3).

When the formulations were compared with respect to the capability of reducing parasite load, the MA formulation in mixture of pegylated liposomes and conventional liposomes proved to be significantly more effective in the spleen, with respect to the formulations of pegylated or conventional liposomes (FIG. 2), and in the bone marrow with respect to the formulation of conventional liposomes (FIG. 3). On the other hand, here was no difference in efficacy between the MA/AM formulations in reducing the parasite load in the liver (FIG. 1).

This study establishes the superiority of the mixture of pegylated liposomes and conventional liposomes with respect to the individual formulations, in reducing the parasite load ion the spleen and in the bone marrow in a model of visceral leishmaniasis.

1-7. (canceled)

8. A pharmaceutical composition for treatment of visceral leishmaniasis, characterized by comprising association of conventional liposomes and prolonged-circulation liposomes, incorporating one or more leishmanicidal drugs.

9. The pharmaceutical composition of claim 8, characterized in that the conventional liposomes comprise natural and/or synthetic phospholipid(s) and/or surfactant(s) and/or cholesterol and have an average diameter ranging from 50 nm to 1000 nm.

10. The pharmaceutical composition of claim 8, characterized in that the prolonged-circulation liposomes include a lipid or surfactant that results in increased time of circulation of the liposomes in the blood stream.

11. The pharmaceutical composition of claim 10, characterized in that the lipid or surfactant has its polar head formed of ethyleneglycol polymer.

12. The pharmaceutical composition of claim 8, characterized in that the one or more leishmanicidal drugs are selected from the group consisting of antiomonial compounds, amphotericin B, pentamidin, miltefosine, alopurinol, paromomycin, sitamaquin, sitamaquine, iminoquimod, fluconazole, cetoconazol, itraconazol, posaconazol, tucaresol, azitromicin, buparvaquone, tamoxifen, terbinafin, furazolidone, fluoroquinolone, domperidone, interleukin 12, interleukin 10, lipid A, derivatives of alkyl-lisophospholipid and of alkyl-phospholipide, azasterols, lichocalcone A, maesabadid III, trichothecenes, N-acetylcysteine, 3-substituted quinolin, and/or other drugs usually employed for treatment of leishmaniasis.

13. The pharmaceutical composition of claim 8, which is formulated for administration by intramuscular, subcutaneous, intraperitoneal, and/or intravenous route.

14. A method for treatment of a form of leishmaniasis in a mammal, the method comprising administering a pharmaceutical composition, which is characterized by association of conventional liposomes and prolonged-circulation liposomes that incorporate one or more leishmanicidal drugs.

15. The method according to claim 14, wherein said conventional liposomes comprise natural and/or synthetic phospholipid(s) and/or surfactant(s) and/or cholesterol and have an average diameter ranging from 50 nm to 1000 nm.

16. The method according to claim 14, wherein said prolonged-circulation liposomes include a lipid or surfactant that results in increased time of circulation of the liposomes in the blood stream.

17. The method according to claim 16, wherein said lipid or surfactant has its polar head formed of ethyleneglycol polymer.

18. The method according to claim 14, wherein said one or more leishmanicidal drugs are selected from the group consisting of antiomonial compounds, amphotericin B, pentamidin, miltefosine, alopurinol, paromomycin, sitamaquin, sitamaquine, iminoquimod, fluconazole, cetoconazol, itraconazol, posaconazol, tucaresol, azitromicin, buparvaquone, tamoxifen, terbinafin, furazolidone, fluoroquinolone, domperidone, interleukin 12, interleukin 10, lipid A, derivatives of alkyl-lisophospholipid and of alkyl-phospholipide, azasterols, lichocalcone A, maesabadid III, trichothecenes, N-acetylcysteine, 3-substituted quinolin, and/or other drugs usually employed for treatment of leishmaniasis.

19. The method according to claim 14, wherein said pharmaceutical composition is administered by intramuscular, subcutaneous, intraperitoneal, and/or intravenous route.
